(19) United States
(12) Patent Application Publication
(10) Pub. No.: US 2024/0343685 A1
(43) Pub. Date: Oct. 17, 2024

(54) SYSTEM FOR PURIFICATION OF ACRYLAMIDO TERTIARY BUTYL SULFONIC ACID AND PROCESS THEREOF

(71) Applicant: VINATI ORGANICS LIMITED, Mumbai (IN)

(72) Inventor: Vinod Kumar Saraf, Mumbai (IN)

(21) Appl. No.: 18/757,547

(22) Filed: Jun. 28, 2024

Related U.S. Application Data
(63) Continuation of application No. PCT/IB2022/051352, filed on Feb. 16, 2022.

(30) Foreign Application Priority Data
Dec. 31, 2021 (IN) 202121062115

(51) Int. Cl. C07C 303/44; B01D 33/06; B01D 33/46; B01D 33/60; B01D 33/66

(52) U.S. Cl. CPC C07C 303/44; B01D 33/06; B01D 33/466; B01D 33/60; B01D 33/66; B01D 2201/202

(57) ABSTRACT

A system (100) for purification of acrylamido tertiary butyl sulfonic acid monomer (ATBS) and process (400) thereof are disclosed herein. The said system (100) may comprise a continuous rotary pressure filter (102), a continuous dryer (103), a product cooler (104), a granulation unit (105) comprising a roller compactor (106), a granulator (107), a sifter (108) and a powder transfer system (PTS) (109). The said continuous rotary pressure filter (102) comprises a scrapper (203) and a rotating filter drum (204). The said rotating filter drum (204) further comprises plurality of filter cells (205). The pressure applied in the continuous rotating pressure filter (102) enables maximum filtration of filtrate from wet cake of ATBS monomer and thereby producing the ATBS product with reduced yellowness. The powder transfer system (PTS) (109) comprises at least four valves controlled to enable the production of high yield of ATBS with uniform particle size and shape.

### CROSS-REFERENCE TO RELATED APPLICATIONS AND PRIORITY

The present application is a continuation application of PCT application No. PCT/IB2022/051352 filed on Feb. 16, 2022, which claims priority from Indian complete patent application No. 202121062115 filed on Dec. 31, 2021. The details of all of the aforementioned applications are incorporated by reference herein in their entirety.

### TECHNICAL FIELD

The present subject matter described herein, in general, relates to a system for the purification of acrylamido tertiary butyl sulfonic acid (ATBS) monomer and a process thereof.

### BACKGROUND

Acrylamido tertiary butyl sulfonic acid monomer (hereinafter referred to as “ATBS”) is a white, needle-like crystal at a normal state and has a melting point of 185° C. ATBS has wide applications in the fields of oil industry, mineral industry, construction, water treatment, fibres, plastics, printing and dyeing, coating, static inhibitor, pottery, washing auxiliary detergent, ion exchange, gas separations and cosmetics.

In current state of art, the processes for the synthesis of ATBS involves a first step of preparation of sulfonating mixture, a second step of the reacting the sulfonating mixture prepared in first step with excess of acrylonitrile (ACRN) in controlled temperature and pressure conditions to obtain ACRN-sulfate. Further, the ACRN-sulfate obtained in the second step is reacted with Isobutylene (IB) to obtain ATBS slurry. The ATBS slurry obtained can be further filtered and purified to remove impurities from the final product. ATBS can be represented by the following formula,

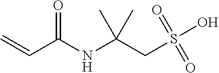

In the existing art, in the step of purification of ATBS monomer slurry various filtration methods are used. However, such conventional processes of using filtration assemblies and methods for the filtration of ATBS monomer suffers from several drawbacks or challenges. Firstly, during the preparation of the sulfonating mixture by using existing methods of filtration yield of ATBS is low. Further, use of existing processes for the filtration of ATBS slurry results in production of yellow colored product with non-uniform particle size and shape, thereby affecting the overall quality the end product. It should be noted that filtration of the ATBS slurry is significant in order to achieve good quality of the ATBS monomer. Further, the system and process as disclosed in the present invention gives higher yield of ATBS monomer by reducing impurities, and by-product content and producing the ATBS with uniform particle size and shape. The impurities present in the ATBS strongly affects, the molecular weight of the ATBS during polymerization and hence further to its usage.

Therefore, there is a long felt need of providing an improved system and process for the purification of ATBS and a preparation process for the purification thereof in order to alleviate at least the aforementioned drawbacks of the existing processes.

### OBJECTS OF THE INVENTION

The principal object of this invention is to provide a system for purification of ATBS enabled to provide high yield and high purity of ATBS.

Another object of this invention is to provide a continuous process for the purification of ATBS enabled to provide high yield and high purity of ATBS.

Another object of this is to invention provide a system and process enabled to produce ATBS with reduced amount of impurities and by-products.

Another object of this invention is to provide filtration assembly and method for the filtration of ATBS slurry enabled to reduce the filtration time of ATBS slurry.

### SUMMARY

This summary is provided to introduce concepts related to a system and process for purification of acrylamido tertiary butyl sulfonic acid monomer (ATBS). This summary is not intended to identify essential features of the claimed subject matter, nor it is intended for use in determining or limiting the scope of the disclosed subject matter.

In accordance with an embodiment of the present subject matter, a system and process for the purification of acrylamido tertiary butyl sulfonic acid monomer (ATBS) is described herein.

In one embodiment, the said system may comprise a continuous rotary pressure filter comprising a rotating filter drum enabled to filter an ATBS slurry to obtain wet cake of ATBS monomer. Further, the said system may comprise a continuous dryer enabled to remove any liquid remaining in the wet cake of ATBS monomer and to obtain a dry powder of ATBS monomer. Further, the said system may comprise a product cooler enabled to cool the dry powder of ATBS monomer to room temperature. Further, the said system may comprise a granulation unit enabled to produce ATBS monomer granules of a predetermined size.

In another embodiment, a process for purification of ATBS slurry is disclosed. The process of purification of ATBS slurry may comprise various steps. The said process may comprise a step of filtration of ATBS slurry by a continuous rotary pressure filter and to obtain wet cake of ATBS monomer with predetermined thickness. Further, the said process may comprise a step of drying the wet cake of ATBS monomer in a continuous dryer by applying predetermined temperature to obtain a dry powder of ATBS monomer. Further, the said process may comprise a step of cooling the dry powder of ATBS monomer within the range of 55-60° C. in a product cooler. Further, the said process may comprise a step of producing the granules of ATBS monomer of a predetermined size by a granulation unit.

### LIST OF ABBREVIATIONS

ATBS—Acrylamido tertiary butyl sulfonic acid
AM—Acrylamide
ACRN—Acrylonitrile
IBDSA—Isobutyl disulfonic acid
IBSA—Isobutyl sulfonic acid
TBA-Tertiary butyl acrylamide
AMPDSA—Acrylamido methyl propane disulfonic acid
PTS—Powder transfer system
HPLC—High performance liquid chromatography
LOD—Loss on drying
RPH—Revolution per hour

### BRIEF DESCRIPTION OF DRAWINGS

The detailed description is described with reference to the accompanying Figures. In the Figures, the left-most digit(s) of a reference number identifies the Figure in which the reference number first appears. The same numbers are used throughout the drawings to refer like features and components.

FIG. 1 depicts a system (100) for the purification of acrylamido tertiary butyl sulfonic acid monomer (ATBS), in accordance with an embodiment of the present subject matter.

FIG. 2a depicts a side view of a continuous rotary pressure filter (102) for the purification of acrylamido tertiary butyl sulfonic acid monomer (ATBS), in accordance with an embodiment of the present subject matter.

FIG. 2b depicts a front view of a continuous rotary pressure filter (102) for the purification of acrylamido tertiary butyl sulfonic acid monomer (ATBS), in accordance with an embodiment of the present subject matter.

FIG. 3 depicts a powder transfer system (109) for the purification of acrylamido tertiary butyl sulfonic acid monomer (ATBS), in accordance with an embodiment of the present subject matter.

FIG. 4 depicts a process (400) for the purification of acrylamido tertiary butyl sulfonic acid monomer (ATBS), in accordance with an embodiment of the present subject matter.

FIG. 5 depicts a step of filtration (401) of ATBS slurry by continuous rotary filter (102), in accordance with an embodiment of the present subject matter.

FIG. 6 depicts a step of producing (404) the granules of ATBS monomer, in accordance with an embodiment of the present subject matter.

FIG. 7 depicts a step of recycling (604) of the uncompacted ATBS monomer to roller compactor (106), in accordance with an embodiment of the present subject matter.

FIG. 8 depicts a nitrogen recovery system (202) applied to the continuous rotary pressure filter (102), in accordance with an embodiment of the present subject matter.

### DETAILED DESCRIPTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the particular features, structures or characteristics may be combined in any suitable manner in one or more embodiments.

The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. It must also be noted that, the singular forms “a,” “an,” and “the” include plural references unless the context clearly dictates otherwise. Although any methods similar or equivalent to those described herein can be used in the practice or testing of embodiments of the present disclosure, the exemplary methods are described. The disclosed embodiments are merely exemplary of the disclosure, which may be embodied in various forms.

Various modifications to the embodiment may be readily apparent to those skilled in the art and the generic principles herein may be applied to other embodiments. However, one of ordinary skill in the art may readily recognize that the present disclosure is not intended to be limited to the embodiments illustrated but is to be accorded the widest scope consistent with the principles and features described herein. The detailed description of the invention will be described hereinafter referring to accompanied drawings.

In accordance with an embodiment of the present subject matter, a system (100) and process (400) for the purification of acrylamido tertiary butyl sulfonic acid monomer (ATBS) is described herein.

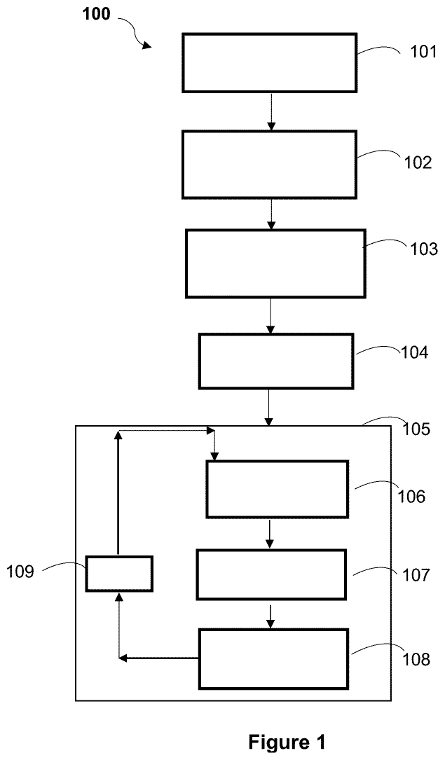

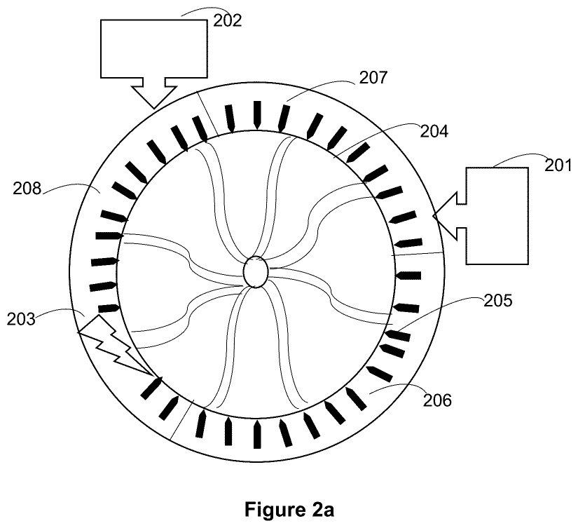

In one embodiment, referring to FIGS. 1, 2a and 2b the said system (100) may comprise a continuous rotary pressure filter (102) comprising a rotating filter drum (204) enabled to filter the ATBS slurry and to obtain wet cake of ATBS monomer.

In one embodiment, referring to FIGS. 2a and 2b, the said continuous rotatory pressure filter (102) may comprises at least three units comprising a filtration unit (206), a washing unit (207) and a drying unit (208).

In one embodiment, the said rotating filter drum (204) may be divided by circumferential and longitudinal segments into a plurality of individual filter cells (205).

In one embodiment, the said plurality of filter cells (205) may be varied between 100-120 and more particularly the number of filter cells (205) are 108. In one embodiment, the shape of filter cells may be circular, triangular, and rectangular. In a preferred embodiment, the shape of filter cells may be rectangular having a length of 400 mm and a breadth of 109 mm. The plurality of filter cells (205) may be configured to produce the wet cake of ATBS monomer with predetermined thickness.

In one embodiment, the filter cells (205) are configured to act as a separating element between the filtrate and ATBS crude granules. In an embodiment, an ATBS crude slurry is fed on a filter cloth (not shown in figure) of the filter cells (205) and due to the pressure difference across a filter medium, an ATBS wet cake is deposited on a front side of the filter cloth and ACRN filtrate is separated through a filtrate pipe from back side of the filter cloth.

In one embodiment, the said rotating filter drum (204) may rotate concentrically, at predetermined speed in a pressure tight housing (not shown in figure), wherein the said pressure tight housing may be configured to said filter drum (204) and the said pressure tight housing may be sealed by special stuffing boxes and divided into pressure-tight zones by separating elements.

In one embodiment, the said continuous rotary pressure filter (102) may comprise a scrapper (203) enabled to remove the final ATBS monomer dry cake from the rotating filter drum (204).

In one embodiment, the ATBS slurry, from the slurry hold vessel (101), comprising of ATBS along with Acrylonitrile and the by-product is fed to the continuous rotary pressure filter (102) through a slurry inlet (209) at the rate of 9000-9500 kg/hr.

Further, the said ATBS slurry is washed with a washing liquid from washing liquid vessel (201) supplied through a washing liquid nozzle (218) to obtain ATBS monomer wet cake. Further, an inert gas such as nitrogen is continuously applied to the continuous rotary pressure filter (102) through a nitrogen gas inlet (210) at a pressure 2.5-3 kg/cm².

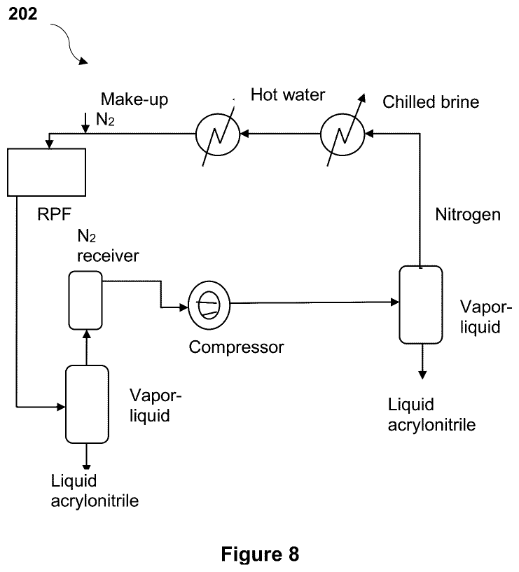

In one embodiment, referring to FIG. 8, the continuous rotary pressure filter (102) may comprise a nitrogen recovery and recycle unit (202) described in detail hereinafter. A constant pressure by nitrogen is applied to the filter cells (205) to carry out the filtration. The said pressure may be maintained at a constant level by implementing the nitrogen recovery and recycle unit (202). The nitrogen recovery and recycle unit (202) may comprise a compressor, vapor-liquid separator tanks, cooler and heater. Further, the nitrogen from an outlet nozzle (215) may be connected to two vapor liquid separator tanks where liquid acrylonitrile is separated from bottom and nitrogen gas is fed from top. The separated nitrogen may be connected to suction of the compressor. The compressor may compress the nitrogen gas up to 3 kg/cm²(g) pressure and the compressed nitrogen may be passed through cooler followed by the heater unit. A hot nitrogen from the heater unit may then be passed to the continuous rotatory pressure filter (102). In one embodiment, the recycle of nitrogen may be continued keeping the constant pressure.

The continuous rotary pressure filter (102) is rotated by means of electrical motor (216), wherein the said electric motor (216) further comprises a rotating shaft (217) which rotates at a speed of 50-55 RPH. The pressure in the continuous rotary pressure filter (102) is maintained between 2.5-3 kg/cm². Due to this positive pressure applied on the wet cake of ATBS, the filtrate is pushed through the filter cloth and the wet cake of ATBS is deposited on the surface of filter cloth. The wet cake of ATBS is continuously scrapped by the scrapper (203) and fed to the rotary vacuum paddle dryer (103) through a cake discharge outlet (211) via a screw conveyor (not shown in figure). Mother filtrate is removed through a mother filtrate nozzle (212). The washing liquid such as acrylonitrile is removed through a washing filtrate nozzle (213). Rest filtrate is removed from the rest filtrate nozzle (214). Further, the outlet nozzle (215) is provided for the aeration and leakage.

Further, the continuous rotary pressure filter (102) may also comprise an earth lug (219) which provides earthing to the overall system of the continuous rotary pressure filter (102). Further, the earth lug (219) is enabled to absorb the static charge generated due to flow of acrylonitrile.

In an embodiment, the continuous rotary pressure filter (102) may comprise a vent end shield (221) acting as a vent in case of any leakage in pressurized system through glands of the chambers is observed. Further, an exhaust nozzle (220) may be configured for escaping of a blow back nitrogen fed to a back side of filter clothes of the filter cells (205) for cake lifting purpose, wherein the blow back nitrogen may be configured to fed through the blow back nozzle (224). The exhaust nozzle (220) as shown in FIG. 2b may be further connected to a common vent line (not shown in figure).

In an embodiment, the said a continuous rotary pressure filter (102) may comprise a spray nozzle (222) comprising a view glass and a lamp fitted on the view glass for a clear view of interior activity of the cake discharge zone through a main view glass unit (223).

In one embodiment, the said continuous rotary pressure filter (102) may have a pore size between the range of 10-20 micron, and preferably between 12-16 micron.

In one embodiment, the said system may comprise a continuous dryer (103) enabled to remove any liquid remaining in the wet cake of ATBS monomer and to obtain a dry powder of ATBS monomer. In one embodiment, the said continuous dryer (103) may be a rotary vacuum paddle dryer configured for drying (402) the wet cake of ATBS monomer.

In one embodiment, the said system may comprise a product cooler (104) enabled to cool the dry powder of ATBS monomer to room temperature. In one embodiment, the said the product cooler (104) may be enabled to cool the ATBS monomer powder within the range of 55-60° C.

In one embodiment, the said system may comprise a granulation unit (105) enabled to produce the ATBS monomer granules of a predetermined size. In one embodiment, the size of granules of ATBS monomer is within a range of 0.2 mm to 8 mm, and more preferably 0.8 mm to 5 mm sieve size.

In one embodiment, the said granulation unit (105) may comprise a roller compactor (106) enabled to compact the dry powder of ATBS in the form of sheets or rods. Further, the said granulation unit (105) may comprise a granulator (107) enabled to produce ATBS monomer granules from the compacted dry powder of ATBS. The said granulation unit (105) may further comprise a sifter (108) enabled to separate the ATBS monomer granules and powder of ATBS monomer slipped through the roller compactor (106) and powder formed in the granulator (107).

Furthermore, the said granulation unit (105) may comprise a powder transfer system (PTS) (109) enabled to recycle the uncompacted ATBS monomer powder to the roller compactor (106). In one embodiment, the said roller compactor (106) may comprise at least two press rollers driven by geared motors (not shown in figure).

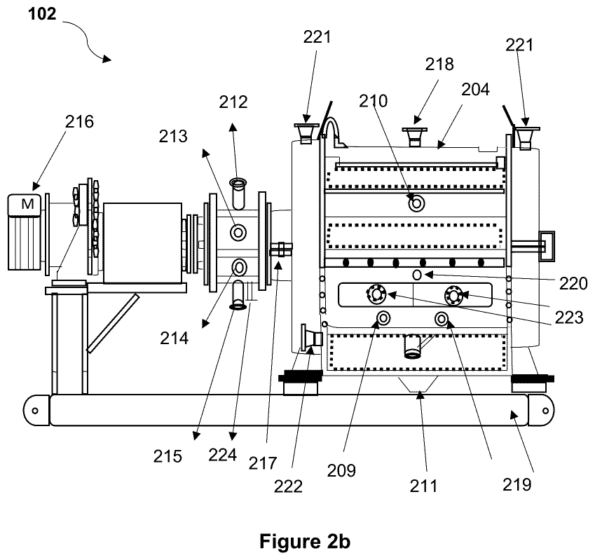

In one embodiment, referring to FIG. 3, the said powder transfer system (PTS) (109) may comprise at least four valves V1 (301), V2 (302), V3 (303), and V4 (304) enabled to suck the uncompacted ATBS powder back using vacuum and recycle the uncompacted ATBS powder back to the roller compactor (106).

In an embodiment, the PTS (109) system may be configured to recycle back the ATBS powder back to compactor by means of PTS system. Referring to FIG. 3, the PTS (109) may comprise of a conical shaped metallic pot (305) connected to ON-OFF control valves V1 (301), V2 (302), V3 (303), and V4 (304). The control valves may be configured to control one or more functions such as, V1 (301) may be configured for applying vacuum to the PTS (109) with a time interval of 10-20 seconds and preferably 12 seconds. Further, the valve V2 (302) may be configured for ATBS powder sucking from the sifter (108) with a time interval of 8-12 seconds and preferably seconds. The valve V3 (303) may be configured for nitrogen purging with a time interval of 1 second and the valve V4 (304) may be configured for powder discharge to roller compactor (106).

In one embodiment, an opening and closing of the control valves 301, 302, 303 and 304 may be configured to be controlled through a distributed control system (DCS) such that the control valves may be configured to open and close for a certain time interval in a sequential manner.

In one embodiment, the sequence of opening and closing of the control valves is as follows. Valve V1 (301) may be opened first, and it remains opened for 12 seconds. Further, Valve V2 (302) may be opened after 2 seconds of opening of the valve V1 (301) and wherein the valve V2 (302) may remain opened for 10 seconds. Further, in the duration of these 10 seconds the powder from the sifter (108) may be sucked to the metallic pot (305), and a simultaneous closing of both the valves V1 (301) and V2 (302) may be carried out. In one embodiment, the valve V3 (303) may be configured to be opened for a time interval of 1 second and then the valve V4 (304) may be configured for simultaneous opening and discharging the powder in a silo storage, wherein the valve V4 (304) may be closed after 6 seconds. In one embodiment, a controlled working of the control valves of the PTS (109) may be continued for a plurality of times.

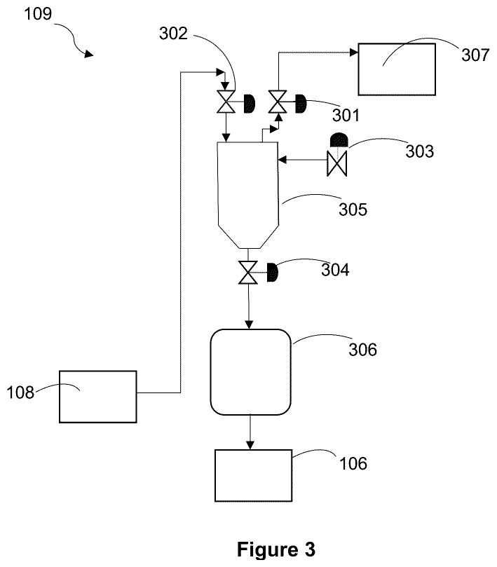

In another embodiment, referring to FIG. 1, FIG. 2a, and FIGS. 2b, and 4, a process (400) for purification of ATBS is disclosed. The process (400) of purification of ATBS may comprise various steps. The said process (400) may comprise a step of filtration (401) of ATBS slurry by the continuous rotary pressure filter (102) and to obtain a wet cake of ATBS monomer. Further, the said process (400) may comprise a step of drying (402) the wet cake of ATBS monomer in the continuous dryer (103) by applying predetermined temperature to obtain a dry powder of ATBS monomer. Further, the said process (400) may comprise a step of cooling (403) the dry powder of ATBS monomer within the range of 55-60° C. in the product cooler (104). Further, the said process (400) may comprise a step of producing (404) the granules of ATBS monomer of a predetermined size by the granulation unit (105).

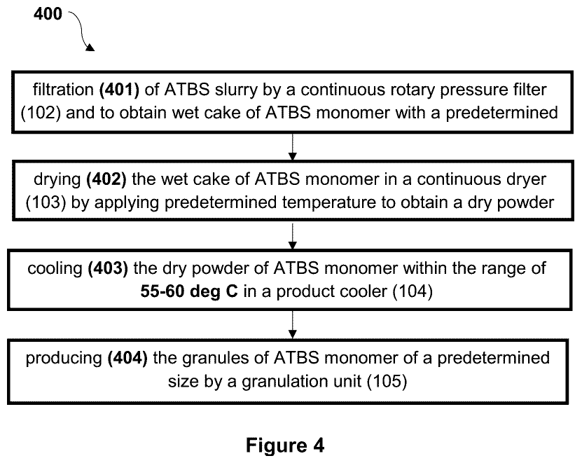

In one embodiment, referring to FIGS. 4 and 5, the said process (400) comprising the step of filtration (401) further may comprise various steps. The said step of filtration (401) may comprise a step of feeding (501) the ATBS slurry from the slurry hold vessel (101) to the continuous rotary pressure filter (102) through a slurry inlet (209). Further, the said step of filtration (401) may comprise a step of washing (502) the ATBS slurry with a washing liquid supplied from a washing liquid vessel (201) through a washing liquid nozzle (218) to obtain ATBS monomer wet cake. Further, the said step of filtration (401) may comprise a step of applying (503) a constant pressure over a filter media comprising wet cake of ATBS monomer by passing an inert gas such as nitrogen from the nitrogen recovery and recycle unit (202) through a nitrogen gas inlet (210) in the drying unit (208).

Further, the said step of filtration (401) may comprise a step of removing (504) the wet cake of ATBS monomer with the help of the scrapper (203). Further, the step of filtration (401) may comprise a step of feeding (505) the wet cake of ATBS monomer to the continuous dryer (103) through a cake discharge outlet (211) via screw conveyor (not shown in figure). In an embodiment, the said continuous dryer (103) may be a rotary vacuum paddle dryer (103).

In one embodiment, the rate of feeding may be 9000-9500 kg/hr in the step of feeding (501).

In another embodiment, the said continuous rotating pressure filter (102) may be rotating at an optimized speed of 50-55 RPH.

In another embodiment, in the step of washing (502), the washing liquid may be an acrylonitrile.

In another embodiment, in the step of applying (503) a constant pressure, the constant pressure may be optimized between 1-10 kg/cm²(g). In another embodiment, the constant pressure may be applied to the continuous rotary pressure filter (102) for maximum draining out of an acrylonitrile filtrate.

In another embodiment, in the step of drying (402), the predetermined temperature maybe in between the range of 100-120° C.

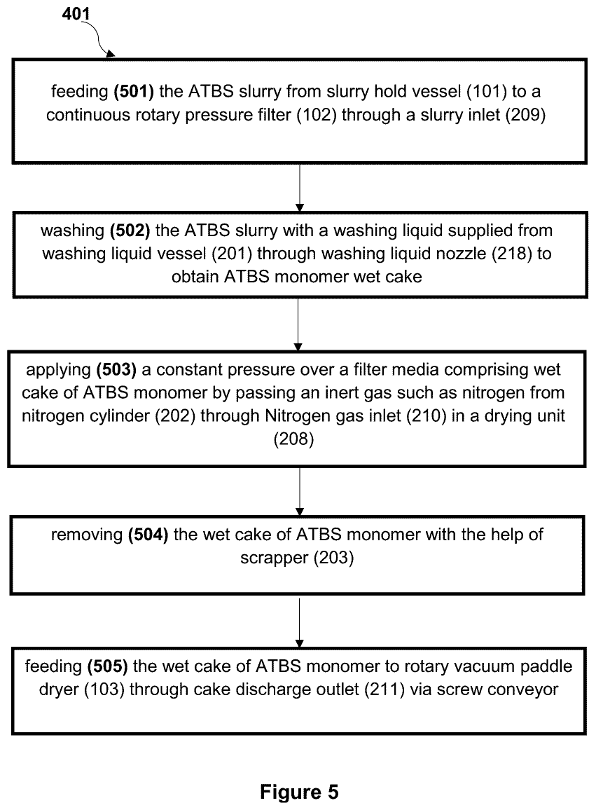

In one embodiment, referring to FIGS. 4 and 6, the said process (400) comprising step of producing (404) the granules of ATBS monomer may comprise various steps. The said step of producing (404) the granules of ATBS monomer may comprise a step of compacting (601) the dry powder of ATBS monomer by using the roller compactor (106) to obtain powdered ATBS in the form of sheets or rods. Further, the said step of producing (404) the granules of ATBS monomer may comprise a step of granulating (602) the powder of ATBS monomer by using the granulator (107) to obtain granules of ATBS monomer. Further the said step of producing (404) the granules of ATBS monomer may comprise a step of sifting (603) the granules of ATBS monomer by using the sifter (108) to obtain granules of ATBS monomer with uniform size and shape. Further the said step of producing (404) the granules of ATBS monomer may comprise a step of recycling (604) of the uncompacted ATBS monomer through the powder transfer system (PTS) (109) to the roller compactor (106).

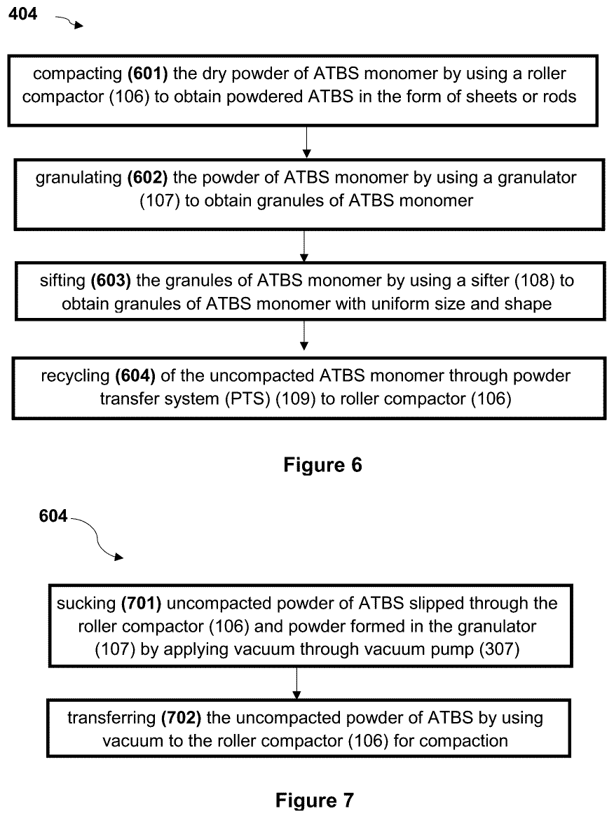

In one embodiment, referring to FIGS. 4, 6 and 7, the said process (400) comprising step of recycling (604) of the uncompacted ATBS monomer may comprise various steps. The said step of recycling (604) may comprise a step of sucking (701) uncompacted powder of ATBS slipped through the roller compactor (106) and powder formed in the granulator (107) by applying vacuum through a vacuum pump (307). Further, the said step of recycling (604) may comprise a step of transferring (702) the uncompacted powder of ATBS by using a vacuum to the roller compactor (106) for compaction.

In another embodiment, the thickness of wet cake of ATBS may be set between 1 to 50 mm, preferably between 6 to 24 mm.

In another embodiment, the purified ATBS monomer may comprise impurities including, Acrylamide (AM) within a range of 610-670 ppm, Acrylonitrile (ACRN) within a range of 180-250 ppm, Isobutyl disulfonic acid (IBDSA) within a range of 35-75 ppm, Isobutyl sulfonic acid (IBSA) within a range of 50-80 ppm, Tertiary butyl acrylamide (TBA) within a range of 1500-1600 ppm and Acrylamido methyl propane disulfonic acid (AMPDSA) within a range of 0.20-0.50%.

In another embodiment, the yield of ATBS monomer may be at least 79-81% and purity of ATBS may be within a range of 98-99.50%.

In another embodiment, the loss on drying of the final ATBS product may be between 10-20%, preferably between 12-18%.

In another embodiment, loss on drying enables reduction in the scaling of a continuous dryer (102), reduction in heat load and washing time of continuous dryer (103) during step of drying (402).

In another embodiment, the pressure applied in the continuous rotating pressure filter (102) enables maximum filtration of filtrate from wet cake of ATBS monomer.

In one embodiment, the said system (100) and process (400) is enabled to produce high yield of ATBS with improved quality.

The instant subject matter is further described by the following examples:

### Experimental Details

### Example 1: Preparation of ATBS Monomer by Using Belt Filter

In one embodiment, a process for preparation of ATBS monomer by using conventional Belt filter is disclosed herein. In the said process the ATBS slurry from the slurry hold vessel comprising ATBS along with Acrylonitrile and the by-products is fed to Belt filter at the rate of 9000-9500 kg/hr. The vacuum is applied to belt filter in the range of 300-390 mmHg by means of a vacuum pump thereby generating negative pressure inside the belt filter. The filter cloth is allowed to move and stop for a particular time interval of seconds. Due to vacuum, the filtrate is sucked from the bottom of the filter cloth and collected in the vessel.

The wet cake of ATBS gets deposited on the top surface of the filter cloth. As the belt moves forward, the wet cake of ATBS also moves and at the extreme end it is scrapped by a scrapper. Meanwhile during the movement of the cake within the belt filter washing of Acrylonitrile is given continuously at the rate of 3500-4500 kg/hr to remove the impurities from the wet cake of ATBS.

As this wet cake of ATBS is cut-off at the extreme end, the said wet cake of ATBS falls down to rotary dryer through the hopper continuously. Then it is continuously dried in the rotary vacuum paddle dryer and fed to roller compactor, granulator and sifter assembly to obtain granules of ATBS.

### Example 2: Preparation of ATBS Monomer by Using Continuous Rotary Pressure Filter

In another exemplary embodiment by referring to FIG. 1-4, a system (100) and a process (400) for the purification of ATBS monomer is disclosed herein. The said system (400) comprises the continuous rotary pressure filter (102) for the filtration of ATBS slurry. The said ATBS slurry from the slurry hold vessel (101) comprising of ATBS along with Acrylonitrile and the by-product is fed to the continuous rotary pressure filter (102) through the slurry inlet (209) at the rate of 9000-9500 kg/hr. Further, the said ATBS slurry is washed with a washing liquid from the washing liquid vessel (201) supplied through the washing liquid nozzle (218) to obtain ATBS monomer wet cake. Further, an inert gas such as nitrogen is continuously applied to the continuous rotary pressure filter (102) through the nitrogen gas inlet (210) at the pressure 2.5-3 kg/cm². The continuous rotary pressure filter (102) is rotated by means of an electrical motor (216), wherein the said electric motor (216) further comprises a rotating shaft (217) which rotates at a speed of 50-55 RPH. The pressure in the continuous rotary pressure filter (102) is maintained between 2.5-3 kg/cm². Due to this positive pressure applied on the wet cake of ATBS the filtrate is pushed through the filter cloth and the wet cake of ATBS is deposited on the surface of filter cloth. The wet cake of ATBS is continuously scrapped by the scrapper (203) and fed to the rotary vacuum paddle dryer (103) through the cake discharge outlet (211) via a screw conveyor (not shown in figure). Mother filtrate is removed through the mother filtrate nozzle (212). The washing liquid such as acrylonitrile is removed through the washing filtrate nozzle (213). Rest filtrate is removed from the rest filtrate nozzle (214). Further, the outlet nozzle (215) is provided for the aeration and leakage.

In an embodiment, Table 1 appended below depicts a composition of the mother filtrate, the rest filtrate and the washing filtrate.

TABLE 1

| Components | IBDSA | AM | IBSA | ACRN | ATBS | TBAA | AMPDSA | % M/C |
|---|---|---|---|---|---|---|---|---|
| % in Mother | 0.34 | 0.20 | 1.90 | 95.22 | 0.34 | 1.90 | 0.01 | 0.09 |
| filtrate | | | | | | | | |
| % in Wash | 0.29 | 0.13 | 0.92 | 92.21 | 5.07 | 1.21 | 0.05 | 0.13 |
| filtrate | | | | | | | | |
| % in rest | 2.69 | 0.42 | 0.90 | 85.24 | 8.80 | 0.61 | 1.24 | 0.10 |
| filtrate | | | | | | | | |

### Example 3: A Comparative Data in Terms of LOD of Wet Cake of ATBS. Quality of Dry ATBS and Output from Combined Filtration and Drying Operation

The wet cake of ATBS is prepared in accordance with the exemplary embodiment 1 and 2. Further, the wet cake of ATBS is dried continuously to obtain dry powder of ATBS and the obtained dry powder is fed to the granulation unit (105) to obtain the granules of ATBS with uniform size and shape. A comparative data in terms of loss of drying (LOD) for conventional belt filter vs a continuous rotary pressure filter is described in the Table 2. A Loss on drying (LOD) of wet cake of ATBS, dry ATBS output and APHA of the final ATBS product was determined for both the belt filter and the continuous rotary pressure filter and the results are tabulated as below:

TABLE 2
A comparative data in terms of LOD of wet ATBS, quality of dry
ATBS and output from combined filtration and drying operation

| | Belt filter | | | | Continuous Rotary Pressure Filter | | | |
|---|---|---|---|---|---|---|---|---|
| | Feed | | Dry ATBS | APHA | Feed | | Dry ATBS | APHA |
| Lot | rate | LOD | output | (Dry | rate | LOD | output | (Dry |
| No. | Kg/hr | % | Kg/hr | ATBS) | Kg/hr | % | Kg/hr | ATBS) |
| Lot-1 | 9514 | 20.16% | 1875 | 17 | 9500 | 15% | 1944 | 18 |
| Lot-2 | 9563 | 19.45% | 1875 | 19 | 9500 | 18% | 1900 | 17 |
| Lot-3 | 9563 | 20.13% | 1875 | 15 | 9538 | 15% | 1925 | 16 |
| Lot-4 | 9671 | 19.92% | 1875 | 16 | 9625 | 14% | 1913 | 16 |
| Lot-5 | 9707 | 19.76% | 1888 | 17 | 9750 | 16% | 2000 | 19 |
| AVG. | 9604 | 19.9% | 1878 | 17 | 9583 | 15.6% | 1936 | 17 |

By referring to table 2, it is evident that the loss on drying was reduced in case of use of the continuous rotary pressure filter in the filtration ofATBS slury while increasing the yield ofthe final ATBS monomer. Also, from the table 2 it is evident that the final ATBS monomer is having required APHA, wherein the APHA is a color standard named for the American Public Health Association and defined by ASTM D1209.

TABLE 3
Comparative summary of results

| Sr. | | | | Rotary pressure |
|---|---|---|---|---|
| No. | Parameter | Unit | Belt filter | filter |
| 1 | Feed rate (Avg.) | kg/hr | 9604 | 9583 |
| 2 | LOD (Avg.) | % | 19.9 | 15.6 |
| 3 | APHA (Avg.) | Number | 17 | 17 |
| 4 | Output, Dry ATBS | kg/hr | 1878 | 1936 |
| | (Avg.) | | | |

Referring to Tables 1-2 and 3, it is evident that the performance of the Continuous Rotary Pressure Filter is observed to be better than the Belt Filter in terms of reduction in LOD and quantitative throughput of ATBS monomer along with the required APHA.

### Example 4: The Effect of Pressure Applied in Continuous Rotary Pressure Filter on Rate of Filtration

In another exemplary embodiment, the effect of pressure applied in rotary pressure filter in accordance with Example 1 and Example 2 is evaluated. The pressure applied on the filter cloth by using an inert gas such as nitrogen is the key factor which increases the rate of filtration in unit time. This pressure value is more than that of negative pressure applied to the belt filter. The applied positive pressure leads to filter out maximum filtrate from the wet cake of ATBS monomer.

The positive pressure kept in the continuous rotary pressure filter is 2.5-3.0 bar whereas the negative pressure kept in belt filter is 300-400 mmHg.

### Example 5: The Effect of Powder Transfer System (PTS) on the Quality and Yield of ATBS Monomer

The system (100) in accordance with the embodiment of the present invention comprises the powder transfer system (PTS) (109) enabled to recycle the uncompacted ATBS monomer to the roller compactor (106). The powder of ATBS slipped through the roller compactor (106) and powder formed in the granulator (107) is sucked by using vacuum and transferred to the roller compactor (106) by means of the powder transfer system (109), thereby reducing the loss of ATBS powder and producing the ATBS monomer with uniform size and shape. Hence the quality and yield of the final ATBS monomer is increased.

By referring to FIG. 3, the working of the powder transfer system (PTS) (109) is provided herein. The function of the said powder transfer system (PTS) (109) mainly depends on the four ON-OFF control valves V1 (301), V2 (302), V3 (303) and V4 (304) as shown in FIG. 3. Each valve is operated i.e., opened and closed with a certain time interval.

The sequence of opening time interval is as follows:
V1 (301): Vacuum—12 sec.
V2 (302): Powder inlet—10 sec.
V3 (303): Nitrogen purging—1 sec.
V4 (304): Powder discharge—6 sec.
V1 (301) and V2 (302) are closed simultaneously, and then V3 (303) and V4 (304) are opened.

The said powder transfer system (PTS) (109) operates in auto mode and can be controlled monitored from the distributed control system (DCS).

### Example 6: Impurity Profile Using Belt Filter and Continuous Rotary Pressure Filter

The commercially important byproduct extracted from the filtration process is Tertiary butyl acrylamide (TBA). TBA content in ATBS product obtained from continuous rotary pressure filter is lesser than that of belt filter. The concentrations of impurities are determined by HPLC, and results are tabulated below:

TABLE 4
Impurity profile while using belt filter
in the preparation of ATBS monomer

| | HPLC, ATBS from belt filter | | | | | |
|---|---|---|---|---|---|---|
| Components | L1 | L2 | L3 | L4 | L5 | AVG. |
| AM, ppm | 686 | 660 | 710 | 647 | 734 | 687 |
| ACRN, ppm | 519 | 416 | 515 | 285 | 437 | 434 |
| IBDSA, ppm | 50 | 55 | 56 | 49 | 55 | 53 |
| IBSA, ppm | 95 | 74 | 67 | 88 | 83 | 81 |
| TBA, ppm | 1690 | 1690 | 1751 | 1743 | 1825 | 1740 |
| AMPDSA % | 0.36 | 0.47 | 0.36 | 0.43 | 0.38 | 0.40 |
| ATBS % | 99.34 | 99.24 | 99.33 | 99.29 | 99.31 | 99.30 |

TABLE 5
Impurity profile while using continuous rotary pressure
filter in the preparation of ATBS monomer:

| | HPLC, ATBS from continuous rotary pressure filter | | | | | |
|---|---|---|---|---|---|---|
| Components | L6 | L7 | L8 | L9 | L10 | AVG. |
| AM, ppm | 594 | 608 | 571 | 614 | 588 | 595 |
| ACRN, ppm | 286 | 209 | 173 | 136 | 152 | 168 |
| IBDSA, ppm | 51 | 54 | 53 | 50 | 51 | 52 |
| IBSA, ppm | 74 | 60 | 54 | 67 | 61 | 61 |
| TBA, ppm | 1459 | 1282 | 1338 | 1446 | 1396 | 1366 |
| AMPDSA % | 0.39 | 0.37 | 0.37 | 0.33 | 0.36 | 0.36 |
| ATBS % | 99.36 | 99.41 | 99.41 | 99.44 | 99.42 | 99.42 |

TABLE 6
Comparative impurity profile while using belt filter and
continuous rotary pressure filter is tabulated below:

| | | | By using |
|---|---|---|---|
| Sr. | Impurity in | By using belt | continuous rotary |
| No. | ATBS | filter | pressure filter |
| 1 | TBA, ppm | 687 | 595 |
| 2 | AM, ppm | 434 | 168 |
| 3 | ACRN, ppm | 53 | 52 |
| 4 | IBSA, ppm | 81 | 61 |

Referring to Tables 4-6, it is evident that the concentration of TBA is reduced in case of use of the continuous rotary pressure filter in the filtration of ATBS slurry as compared to the use of the belt filter in the filtration of ATBS slurry. Further, it can be seen that the impurities like AM, ACRN and IBSA are also reduced. Thus, the purity of ATBS is improved. The reduction in the filtration of impurities lead to the reduction of yellow color of ATBS and a white colored or clear ATBS is obtained with required APHA. The filtration of ATBS slurry by using belt filter produces yellow colored ATBS product.

### Example 7: Effect of Using Belt Filter and Continuous Rotary Pressure Filter on the Yield of ATBS

In another exemplary embodiment, the effect of using the belt filter and the continuous rotary pressure filter on the yield of ATBS is evaluated. The experimental results are tabulated in table 4 and table 5.

By referring to table 4 and 5, it is evident that the percentage yield of ATBS is within the range of 99.2-99.3% while using the belt filter for the filtration of ATBS slurry and the percentage yield of ATBS is within the range of 99.3-99.5% while using the continuous rotary pressure filter for the filtration of ATBS slurry.

In another embodiment, the said system (100) and process (400) may be enabled to decrease the production of by-products in the synthesis of ATBS, wherein the said by-products may be a tertiary butyl acrylamide (TBA) formed during the process of ATBS synthesis.

In another embodiment of the invention, the said system (100) and process (400) may be enabled to decrease the amount of impurities in the synthesized ATBS, wherein the said impurities may be one or more of Acrylamide (AM), acrylonitrile (ACRN), and Isobutyl sulfonic acid (IBSA).

In another embodiment of the invention, the said system (100) and process (400) may be enabled to obtain the ATBS monomer with reduced yellowness and to obtain white colored ATBS monomer with high yield.

The embodiments, examples and alternatives of the preceding paragraphs or the description and drawings, including any of their various aspects or respective individual features, may be taken independently or in any combination. Features described in connection with one embodiment are applicable to all embodiments, unless such features are incompatible.

Although implementations for a system (100) and process (400) for the purification of acrylamido tertiary butyl sulfonic acid (ATBS) have been described in language specific to structural features and/or processes, it is to be understood that the appended claims are not necessarily limited to the specific features or processes described. Rather, the specific features and processes are disclosed as examples of implementations of a system (100) and a process (400) for the purification of acrylamido tertiary butyl sulfonic acid (ATBS).

We claim:

1. A system (100) for purification of ATBS slurry to obtain an ATBS monomer comprising:
a continuous rotary pressure filter (102) comprising a rotating filter drum (204) enabled to filter the ATBS slurry to obtain a wet cake of ATBS monomer;
a continuous dryer (103) enabled to remove any liquid remaining in the wet cake of ATBS monomer and to obtain a dry powder of ATBS monomer;
a product cooler (104) enabled to cool the dry powder of ATBS monomer to room temperature; and
a granulation unit (105) enabled to produce the predetermined size of ATBS monomer granules.

2. The system as claimed in claim 1, wherein the continuous rotatory pressure filter (102) is having pore size of 14 micron, and wherein the continuous rotatory pressure filter (102) comprises at least a filtration unit (206), a washing unit (207) and a drying unit (208).

3. The system as claimed in claim 1, wherein the rotating filter drum (204) is divided by circumferential and longitudinal segments into plurality of individual filter cells (205), wherein the plurality of filter cells (205) are varied between 100-120, and wherein the plurality of filter cells (205) are configured to produce the wet cake of ATBS monomer with a predetermined thickness.

4. The system as claimed in claim 1, wherein the rotating filter drum (204) rotate concentrically, at predetermined speed in a pressure tight housing, wherein the said pressure tight housing may be configured to said filter drum (204) and the said pressure tight housing is sealed by special stuffing boxes and divided into pressure-tight zones by separating elements.

5. The system as claimed in claim 1, wherein the continuous rotary pressure filter (102) comprises a scrapper (203) enabled to remove the final ATBS monomer dry cake from rotating filter drum (204).

6. The system as claimed in claim 1, wherein the continuous dryer (103) is a rotary vacuum paddle dryer configured for drying (402) the wet cake of ATBS monomer.

7. The system as claimed in claim 1, wherein the product cooler (104) is enabled to cool the ATBS monomer powder within the range of 55-60° C.

8. The system as claimed in claim 1, wherein the granulation unit (105) comprises:
a roller compactor (106) enabled to compact the dry powder of ATBS in the form of sheets or rods, wherein the roller compactor (106) comprises at least two press rollers driven by geared motors;
a granulator (107) enabled to produce granules of ATBS monomer from compacted dry powder of ATBS;
a sifter (108) enabled to separate the ATBS monomer granules and a powder of ATBS monomer slipped through the roller compactor (106) and powder formed in the granulator (107); and
a powder transfer system (PTS) (109) enabled to recycle the uncompacted ATBS monomer powder to the roller compactor (106), wherein powder transfer system (PTS) (109) comprises at least four valves V1 (301), V2 (302), V3 (303) and V4 (304) enabled to suck the uncompacted ATBS powder back using vacuum and recycle the uncompacted ATBS powder back to the roller compactor (106).

9. A process (400) of for purification of a ATBS slurry to obtain an ATBS monomer comprising steps of:
filtration (401) of ATBS slurry by a continuous rotary pressure filter (102) and to obtain the wet cake of ATBS monomer with predetermined thickness;
drying (402) the wet cake of ATBS monomer in a continuous dryer (103) by applying predetermined temperature to obtain a dry powder of ATBS monomer;
cooling (403) the dry powder of ATBS monomer within the range of 55-60° C. in a product cooler (104); and
producing (404) the granules of ATBS monomer with predetermined size by a granulation unit (105).

10. The process as claimed in claim 9, wherein the size of granules of ATBS monomer is between the range of 0.2 to 8 mm sieve size.

11. The process (400) as claimed in claim 9, wherein the step of filtration (401) comprises steps of:
feeding (501) the ATBS slurry from slurry hold vessel (101) to a continuous rotary pressure filter (102) through a slurry inlet (209), wherein a rate of feeding is 9000-9500 kg/hr;
washing (502) the ATBS slurry with a washing liquid supplied from washing liquid vessel (201) through washing liquid nozzle (218) to obtain ATBS monomer wet cake, wherein the washing liquid is an acrylonitrile;
applying (503) a constant pressure over a filter media comprising wet cake of ATBS monomer by passing an inert gas such as nitrogen from a nitrogen recovery and recycle unit (202) through nitrogen gas inlet (210) in a drying unit (208), wherein the constant pressure is optimized between 1-10 kg/cm²(g), and wherein the constant pressure is applied to the continuous rotary pressure filter (102) for maximum draining out of an acrylonitrile filtrate;
removing (504) the wet cake of ATBS monomer with the help of scrapper (203); and
feeding (505) the wet cake of ATBS monomer to rotary vacuum paddle dryer (103) through cake discharge outlet (211) via screw conveyor.

12. The process (400) as claimed in claim 9, wherein the continuous rotating pressure filter (102) is rotating at the optimized speed of 50-55 RPH.

13. The process (400) as claimed in claim 9, wherein in the step of drying (402) the predetermined temperature is between the range of 100-120° C.

14. The process as claimed in claim 9, wherein the step of producing (404) the granules of ATBS comprises sub-steps of:
compacting (601) the dry powder of ATBS monomer by using a roller compactor (106) to obtain powdered ATBS in the form of sheets or rods;
granulating (602) the powder of ATBS monomer by using a granulator (107) to obtain granules of ATBS monomer;
sifting (603) the granules of ATBS monomer by using a sifter (108) to obtain granules of ATBS monomer with uniform size and shape; and
recycling (604) of the uncompacted ATBS monomer through powder transfer system (PTS) (109) to roller compactor (106), wherein the step of recycling (604) of the uncompacted ATBS monomer comprises sub-steps of:
sucking (701) uncompacted powder of ATBS slipped through the roller compactor (106) and powder formed in the granulator (107) by applying vacuum through vacuum pump (307); and
transferring (702) the uncompacted powder of ATBS by using vacuum to the roller compactor (106) for compaction.

15. The process (400) as claimed in claim 9, wherein the wet cake thickness is set between 1 to 50 mm.

16. The process (400) as claimed in claim 9, wherein the yield of ATBS monomer is at least 79-81% and purity of ATBS is within a range of 98-99.50%, wherein the loss on drying of the final ATBS product is between 10-20%, and wherein the purified ATBS monomer comprises impurities including:
Acrylamide (AM) within a range of 610-670 ppm;
Acrylonitrile (ACRN) within a range of 180-250 ppm;
Isobutyl disulfonic acid (IBDSA) within a range of 35-75 ppm;
Isobutyl sulfonic acid (IBSA) within a range of 50-80 ppm;
Tertiary butyl acrylamide (TBA) within a range of 1500-1600 ppm; and Acrylamido methyl propane disulfonic acid (AMPDSA) within a range of 0.20-0.50%.
